(19) United States
(12) Patent Application Publication
(10) Pub. No.: US 2005/0059072 A1
(43) Pub. Date: Mar. 17, 2005

(54) Selective modulation of TLR gene expression

(75) Inventors: Woubalem M. R. Birmachu, St. Anthony Village, MN (US); Marla J. C. Burger, Woodbury, MN (US); Raymond M. Gleason, Eagan, MN (US); John A. Hanten, Cottage Grove, MN (US); Jizhong Jin, Vadnais Heights, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/944,291

(22) Filed: Sep. 17, 2004

Related U.S. Application Data
(60) Provisional application No. 60/503,566, filed on Sep. 17, 2003.

(51) Int. Cl. C12Q 1/68; G01N 33/53; G01N 33/567

(52) U.S. Cl. 435/6; 435/7.2

(57) ABSTRACT

The present invention provides a method of identifying a compound that selectively modulates expression of at least one TLR gene. Generally, the method includes providing an assay to detect expression of each of a plurality of TLR genes; performing each assay using a test compound; and identifying the test compound as a compound that selectively modulates expression of at least one TLR gene if the test compound modulates expression of a first TLR gene to a different extent than it modulates expression of at least one second TLR gene. In certain embodiments, the present invention provides compounds identified by a method described above, salts thereof, and pharmaceutical compositions including such compounds, pharmaceutically acceptable forms thereof, derivatives thereof, or pro-drugs thereof.

### CROSS-REFERENCE TO RELATED APPLICATION

This application claims priority to U.S. Provisional Patent Application Ser. No. 60/503,566, filed Sep. 17, 2003.

### BACKGROUND

There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds, referred to herein as immune response modifiers (IRMs), appear to act through immune system mechanisms known as toll-like receptors to induce selected cytokine biosynthesis. They may be useful for treating a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis, melanoma), and TH2-mediated diseases (e.g., asthma, allergic rhinitis, atopic dermatitis, etc.), and are also useful as vaccine adjuvants.

Many of the IRM compounds are small organic molecule imidazoquinoline amine derivatives (see, e.g., U.S. Pat. No. 4,689,338), but a number of other compound classes are known as well (see, e.g., U.S. Pat. Nos. 5,446,153, 6,194,425, and 6,110,929) and more are still being discovered. Other IRMs have higher molecular weights, such as oligonucleotides, including CpGs (see, e.g., U.S. Pat. No. 6,1994,388).

In view of the great therapeutic potential for IRMs, and despite the important work that has already been done, there is a substantial ongoing need to expand their uses and therapeutic benefits.

### SUMMARY

It has been found that certain compounds can selectively modulate expression of certain TLR genes. Accordingly, one aspect of the present invention provides a method of identifying a compound that selectively modulates expression of at least one TLR gene. Generally, the method includes providing an assay to detect expression of each of a plurality of TLR genes; performing each assay using a test compound; and identifying the test compound as a compound that selectively modulates expression of at least one TLR gene if the test compound modulates expression of a first TLR gene to a different extent than it modulates expression of at least one second TLR gene.

In another aspect, the present invention also provides a method of identifying a target compound having a target TLR gene expression profile. Generally, the method includes selecting a target TLR gene expression profile; determining the TLR gene expression profile of a test compound; and identifying the test compound as a target compound if the TLR gene expression profile of the test compound includes the target TLR gene expression profile.

In certain embodiments, the present invention provides compounds identified by a method described above and pharmaceutically acceptable forms thereof, and pharmaceutical compositions including such compounds, pharmaceutically acceptable forms of such compounds, derivatives thereof, or pro-drugs thereof.

In another aspect, the present invention provides a method of modulating expression of a TLR gene in a selected population of cells of the immune system. Generally, the method includes identifying a first immune system cell population and a second immune system cell population; selecting a compound that modulates expression of a TLR gene of the first cell population to a different extent than it modulates expression of the same TLR gene in the second cell population; and contacting cells of the immune system with the selected compound in an amount effective to modulate expression of at least one TLR gene in at least one of the cell populations.

In yet another aspect, the present invention provides a method of treating a condition treatable by selectively modulating expression of at least one of a plurality of TLR genes in a subject. Generally, the method includes identifying a target TLR expression profile effective for treatment of the condition; selecting a compound having a TLR expression profile that conforms to the target profile; and administering to the subject an amount of the compound effective for treating the condition.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 shows modulation of TLR gene expression by IRM1 in PBMCs.

FIG. 2 shows modulation of TLR3 gene expression in PBMCs by IRM compounds.

FIG. 3 shows modulation of TLR7 gene expression in PBMCs by IRM compounds.

FIG. 4 shows modulation of TLR8 gene expression in PBMCs by IRM compounds.

FIG. 5 shows modulation of TLR3 gene expression in macrophages by IRM compounds.

FIG. 6 shows modulation of TLR5 gene expression in macrophages by IRM compounds.

FIG. 7 shows modulation of TLR7 gene expression in macrophages by IRM compounds.

### DETAILED DESCRIPTION OF ILLUSTRATIVE EMBODIMENTS OF THE INVENTION

Immune response modifiers (“IRMs”) include compounds that possess potent immunomodulating activity including but not limited to antiviral and antitumor activity. Certain IRMs modulate the production and secretion of cytokines. For example, certain IRM compounds induce the production and secretion of cytokines such as, e.g., Type I interferons, TNF-α, IL-1, IL-6, IL-8, IL-10, IL-12, MIP-1, and/or MCP-1. As another example, certain IRM compounds can inhibit production and secretion of certain TH2 cytokines, such as IL-4 and IL-5. Additionally, some IRM compounds are said to suppress IL-1 and TNF (U.S. Pat. No. 6,518,265).

Certain IRMs are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; U.S. Patent Publication Nos. 2004/0091491; 2004/0132766; and 2004/0147543; U.S. patent application Ser. No. 10/794,099 filed Mar. 5, 2004; and International Patent Application No. PCT/US04/28021 filed on Aug. 27, 2004.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

It has been found that certain IRMs can selectively modulate expression of Toll-like receptor (TLR) genes. In some cases, selectively modulating TLR gene expression involves modulating expression of one TLR gene, but not significantly modulating expression of another TLR gene. In other cases, selectively modulating TLR gene expression involves modulating expression of one TLR gene in a direction or to an extent that differs from the direction and/or extent to which another TLR gene is modulated.

Accordingly, the present invention provides methods of identifying compounds that selectively modulate TLR gene expression, the compounds thus identified, and pharmaceutical compositions including such compounds; methods of identifying compounds having a particular TLR gene expression profile, the compounds thus identified, and pharmaceutical compositions including such compounds; methods of modulating TLR gene expression in a selected population of immune cells; and methods of treating a subject by administering to the subject a compound that selectively modulates expression of at least one TLR gene.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

For purposes of this invention, the following terms shall have the following meanings.

“Agonist” refers to a compound that can combine with a receptor (e.g., a TLR) to produce a cellular response. An agonist may be a ligand that directly binds to the receptor. Alternatively, an agonist may combine with a receptor indirectly by, for example, (a) forming a complex with another molecule that directly binds to the receptor, or (b) otherwise results in the modification of another compound so that the other compound directly binds to the receptor. An agonist may be referred to as an agonist of a particular TLR (e.g., a TLR7 agonist).

“Express” and variations thereof refer to transcription of mRNA from the structural gene being expressed.

“Immune cell” refers to a cell of the immune system, i.e., a cell directly or indirectly involved in the generation or maintenance of an immune response, whether the immune response is innate, acquired, humoral, or cell-mediated.

“Induce” and variations thereof refer to any measurable increase in gene expression. “Induction” may be used interchangeably with “upregulation.” An “inducer,” therefore, refers to a compound that increases expression of a particular gene.

“Inhibit” and variations thereof refer to any measurable decrease in gene expression. “Inhibition” may be used interchangeably with “suppression” or “downregulation.” An “inhibitor,” therefore, refers to a compound that decreases expression of a particular gene.

“IRM compound” refers generally to a compound that alters the level of one or more immune regulatory molecules, e.g., cytokines or co-stimulatory markers, when administered to an IRM-responsive cell. Representative IRM compounds include the small organic molecules, purine derivatives, small heterocyclic compounds, amide derivatives, and oligonucleotide sequences described above.

“Modulate” and variations thereof refer to any measurable upregulation or downregulation of gene expression.

“Prodrug” refers to a derivative of a drug molecule that requires a chemical or enzymatic biotransformation in order to release the active parent drug in the body.

“Qualitative” and variations thereof refer to (1) the existence (yes/no) of significant modulation of gene expression, (2) the direction (induction/inhibition) of gene expression modulation, or (c) both.

“Quantitative” and variations thereof refer to the magnitude of gene expression modulation without regard to the direction.

“Selective” and variations thereof refer to being able to differentiate between two or more alternatives such as, for example, cell populations, genes, or levels of gene expression. For example, selectively modulating gene expression refers to differentially altering the expression of two or more genes. As another example, modulating gene expression in a selected population of cells refers to modulating expression of a given gene to a particular extent in, for example, one population of cells, and modulating expression of the same gene to a different extent in, for example, a second population of cells.

“TLR gene expression profile” refers to (a) the identity of TLR genes whose expression can be modulated by administration of an IRM, (b) the presence, absence, and/or character of qualitative gene expression modulation, and/or (c) the presence, absence, and/or character of quantitative gene expression modulation. The TLR gene expression profile of a given compound refers to the observed profile of TLR gene expression modulated by the given compound. The observed profile may be compiled from a single source or multiple sources. A target TLR gene expression profile refers to a particular desired profile—which may be, for example, a theoretical or idealized TLR gene expression profile—such as for (a) a target compound to be identified in a screening assay, or (b) for a compound that would modulate TLR gene expression of certain immune cells in a particular manner.

In one aspect, the present invention provides methods of identifying a compound that selectively modulates expression of at least one TLR gene. In general, the methods include providing an assay that can detect expression of each of a plurality of TLR genes; performing each assay using a test compound; and identifying the test compound as a compound that selectively modulates at least one TLR gene if the test compound modulates expression of a first TLR gene to a different extent than it modulates expression of at least one second TLR gene.

The modulation may include upregulation, downregulation, or both. Therefore, certain methods of the present invention could identify compounds that, for example, (a) modulate expression of two or more TLR genes, but do so to varying degrees, or (b) modulate expression of one TLR gene, but do not modulate expression of a second TLR gene. Modulating expression of two or more genes to varying degrees can include, for example, modulating gene expression to different qualitative degrees (e.g., upregulation, downregulation, or no regulation), modulating gene expression in the same qualitative degree, but to different quantitative degrees (e.g., upregulation of one gene more than a second gene), or any combination of quantitative and qualitative degrees.

In some embodiments, at least a two-fold modulation (i.e., upregulation or downregulation) of TLR gene expression may be considered significant. For example, upregulating expression of a TLR gene by at least two-fold may be considered representative of significant modulation of TLR gene expression, while upregulating expression of a TLR gene by less than two-fold may be considered insignificant, for example, as within the scope of experimental error, normal variation, or both. In other embodiments, at least a three-fold modulation of TLR gene expression may be considered significant, while less than a three-fold modulation of TLR gene expression may be considered insignificant. In still other embodiments, at least a four-fold modulation of TLR gene expression may be considered significant, while less than a four-fold modulation of TLR gene expression may be considered insignificant. The precise level of TLR gene expression modulation required to be considered significant may depend, at least in part, on factors including, but not limited to, the intended use of the identified compound (prophylactic, therapeutic, diagnostic, etc.); the quality (e.g., accuracy and/or precision) of the assay used to determine TLR gene expression; and the environment in which the compound is intended to modulate TLR gene expression (e.g., in vitro or in vivo).

Standard techniques are available to one of ordinary skill in the art for designing and performing assays that can detect upregulation and/or downregulation of TLR gene expression. For example, gene expression can be assayed using real-time PCR (RT-PCR), microarray gene analysis, or Northern blot analysis.

Cells used in the assays of the methods of the present invention may be any cells that express one or more TLR genes and permit detection of TLR gene expression. In some cases, the cells may naturally express one or more TLRs. Cells that naturally express one or more TLRs include but are not limited to primary immune cells such as monocytes, macrophages, Langerhans cells, dendritic cells, Natural Killer cells, polymorphonuclear cells (e.g., neutrophils, basophils, or eosinophils), B lymphocytes, T lymphocytes, and cells derived from any of the foregoing.

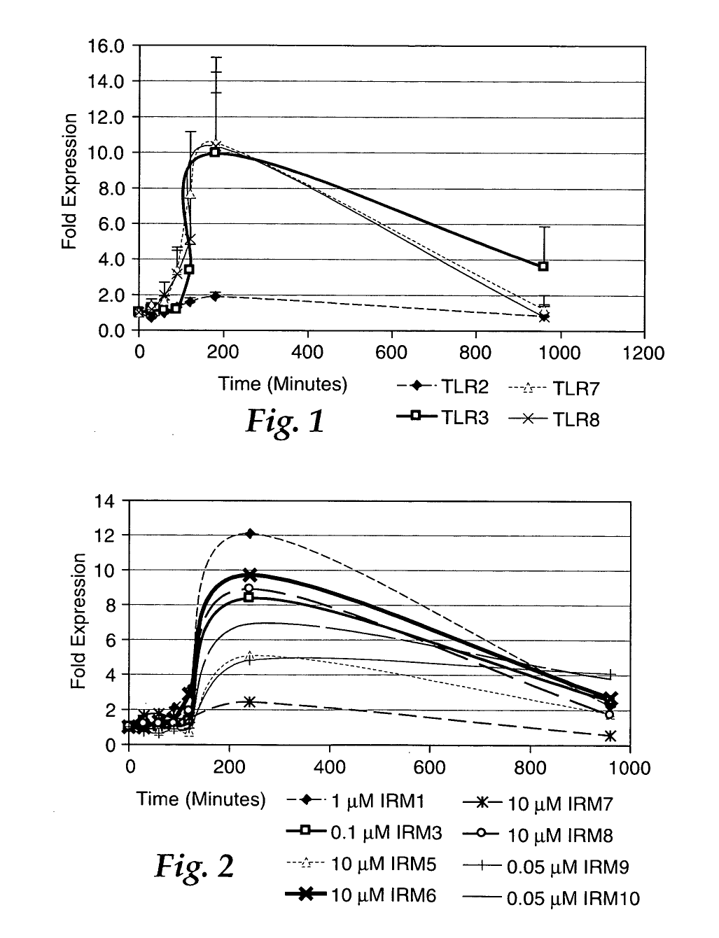

FIG. 1 illustrates selective modulation of TLR gene expression by an IRM compound. Human peripheral blood mononuclear cells (PBMCs) were incubated in vitro with an IRM compound, and expression from each of TLR2, TLR3, TLR4, TLR5, TLR6, TLR7, TLR8, TLR9, and TLR10 was assayed. The IRM compound induces expression of the TLR3 gene, the TLR7 gene, and the TLR8 gene. The IRM compound did not significantly modulate expression from any of the other TLR genes (e.g., TLR2), thereby demonstrating qualitatively selective modulation of TLR gene expression by the IRM compound.

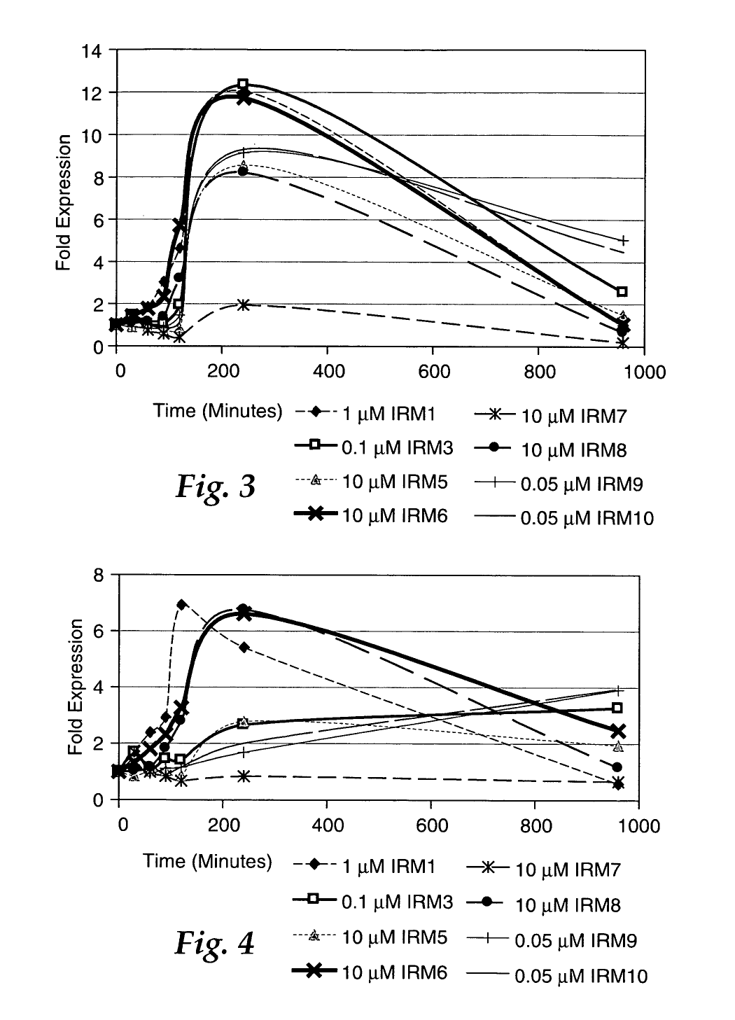

FIGS. 2-4 illustrate the ability of certain IRM compounds to quantitatively modulate expression of a particular TLR gene (e.g., same direction, but to varying degrees). No significant change in TLR gene expression was observed from TLR genes other than TLR3 (FIG. 2), TLR7 (FIG. 3), and TLR8 (FIG. 4). The magnitude of expression at the maximum time point was dependent on the IRM used. The largest IRM-related variation was seen for the expression of TLR8. IRM1, IRM6, and IRM8 give higher and earlier peaks of gene expression than the rest of the compounds tested. IRM7 shows little effect on gene expression from any of TLR3, TLR7, and TLR8.

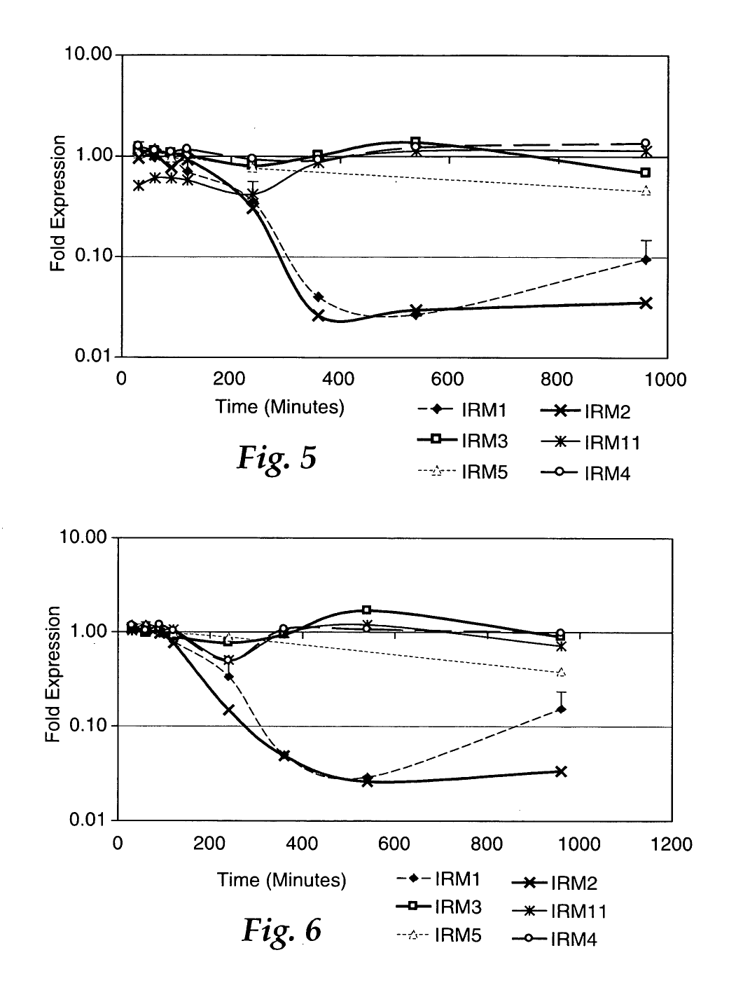

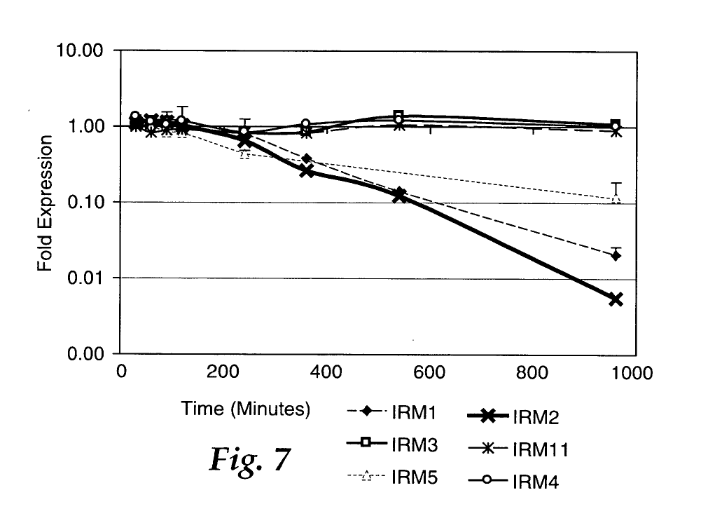

FIGS. 5-7 illustrate that modulating TLR gene expression can include downregulation of gene expression. In macrophages, expression from each of TLR3 (FIG. 5), TLR5 (FIG. 6), and TLR7 (FIG. 7) was downregulated with IRM1 and IRM2.

In dendritic cells, IRM1 may upregulate expression from TLR2, and may downregulate expression from TLR6 and TLR7.

In certain embodiments, the assays may include one or more appropriate controls to ensure that the assays are performing properly. However, one of skill in the art may accumulate sufficient experience and familiarity with, for example, a given assay or the TLR gene expression profile of a particular compound that appropriate controls may not be required each time the assay is performed.

The present invention also provides compounds—and any salts thereof—identified according to the method described above. The methods described above can employ any assay that detects any modulation of expression of any TLR gene. Accordingly, the methods described above can be a powerful tool for identifying a broad spectrum of compounds that selectively modulate the expression of one or more TLR genes. The compounds thus identified may be structurally related to one or more of the various classes of IRM compounds described above. Alternatively, compounds identified by the methods of the present invention may be structurally unrelated to known classes of IRM compounds an d, therefore, may identify a new and previously unknown class of IRM compounds. In either case, such compounds may be incorporated into a pharmaceutical composition. Such pharmaceutical compositions are described in greater detail below.

In another aspect, the present invention provides methods of identifying a target compound having a particular TLR gene expression profile. Generally, the method includes selecting a target TLR gene expression profile; determining the TLR gene expression profile of a test compound; and identifying the test compound as a target compound if the TLR gene expression profile of the test compound conforms to the target TLR gene expression profile.

A target TLR gene expression profile may include one or more TLR genes for which modulation of gene expression is desired (for example, for a prophylactic, therapeutic, or diagnostic effect). For example, a compound that upregulates expression of one or more particular TLR genes may be useful for treating a particular condition. Alternatively, a particular TLR gene expression profile may be useful for identifying either attractive candidates for new drugs, or new uses for known drugs.

A target TLR gene expression profile may include information regarding the TLR gene expression modulating effects of a compound on a plurality of TLR genes. In such embodiments, the TLR gene expression profile may include, in any combination, upregulation, down regulation, and/or no regulation of expression from the selected TLR genes. The particular combination of TLR genes, whether expression from each TLR gene is modulated, and the extent of gene expression modulation for a particular target TLR gene expression profile may at least partially depend upon the particular TLR gene expression characteristics desired for a particular use.

A target TLR gene expression profile may contain as much or as little information as is known and/or required for an intended use. In some cases, the relevant portion of a target TLR gene expression profile may include expression of a single TLR gene, without regard to the expression of any other TLR gene. This may be so because of certain factors including, but not limited to, factors relating to the condition to be treated; the scope of the diagnostic assay being or to be performed; the target cell population whose TLR gene expression (and/or resulting biological activity) is intended to be modulated; the identity of TLR genes being considered and the native level of expression of those genes in the target cells; the location of the target cells—in vitro, in vivo, and if in vivo, the tissue or organ in which the target cells are located; and the general state of the immune system (e.g., suppressed, compromised, stimulated) of a subject.

The TLR gene expression profile of a test compound may be determined in any suitable manner. One method of determining the TLR gene expression profile of a compound is to perform one or more assays such as the assays described above to determine whether a test compound significantly modulates the expression of a particular TLR gene. Alternatively, a particular compound may be known to modulate expression of one or more TLR genes. For example, certain IRM compounds are identified herein as inducers of, for example, the TLR7 gene in peripheral blood mononuclear cells (PBMCs). In some cases, a TLR gene expression profile of a test compound may include information compiled from a plurality of sources.

The TLR gene expression profile of a test compound may contain as much or as little information as is desired for comparison with the target TLR gene expression profile. The extent of the information desired for the TLR gene expression profile of a test compound may depend, at least in part, on a number of factors including but not limited to the factors listed above with respect to the determining the target TLR gene expression profile.

Identifying a test compound as having a particular target TLR gene expression profile involves comparing the TLR gene expression profile of the test compound with the target TLR gene expression profile. In some cases, the target TLR gene expression profile and the TLR gene expression profile of the test compound may form a perfect match. In such cases, the test compound can be readily identified as conforming to the target TLR gene expression profile.

In certain cases in which the target TLR gene expression profile and the TLR gene expression profile of the test compound differ to some extent, the test compound may still be identified as conforming to the desired TLR gene expression profile. For example, in certain cases, qualitative (i.e., the direction of) TLR gene expression modulation may be more important than quantitative (i.e., the magnitude of) modulation. As another example, the test compound might modulate expression of a particular TLR gene that, for the purposes of the target TLR gene expression profile, has little if any relevance. For example, if a target TLR gene expression profile is defined as including induction of TLR7 expression and TLR8 expression in PBMCs, a test compound that induces expression of TLR7, TLR8, and TLR3 in PBMCs may conform to the target TLR gene expression profile, because the induction of TLR3 expression in addition to the desired induction of TLR7 expression and TLR8 expression may not be relevant for a particular application.

The target TLR gene expression profile may vary according to the specific applications for which compounds identified as conforming to the target TLR gene expression profile are to be used. For example, treatment of certain viral infections may benefit from administration of a TLR7 inducer. Such treatments may, for example, increase a treated cell's TLR7-mediated cellular response to a TLR7 agonist such as, for example, production of Type I interferons and activation of certain antigen presenting cells (APCs). Alternatively, treatment of certain types of tumors may benefit from using a compound identified as an inducer of TLR8. Such treatments may, for example, increase a treated cell's TLR8-mediated response to a TLR8 agonist such as, for example, production and/or secretion of IL-12, activation of macrophages, infiltration of the treated area by macrophages, and a strong inflammatory response. Conversely, treatment of certain conditions may benefit from downregulated or suppressed expression of one or more TLR genes in a particular cell population. Such treatments may be useful, for example, for (a) treating certain conditions characterized by chronic inflammation such as rheumatoid arthritis or autoimmune disease, or (b) limiting inflammation due to viral or bacterial infection.

The present invention also provides compounds—and any salts thereof—identified as target compounds according to the method described above. The methods described above can employ any suitable target TLR gene expression profile, incorporating information relating to the expression of any number of TLR genes. Accordingly, the methods described above can be a powerful tool for identifying a broad spectrum of compounds that conform to a particular target TLR gene expression profile. The compounds thus identified may be incorporated into a pharmaceutical composition. Such pharmaceutical compositions are described in greater detail below.

In another aspect, the present invention provides methods of modulating expression of a TLR gene selectively between different populations of cells of the immune system. Generally, the methods include identifying a first immune system cell population and a second immune system cell population; selecting a compound that modulates expression of a TLR gene of the first cell population to a different extent than it modulates expression of the same TLR gene in the second cell population; and contacting cells of the immune system with the selected compound in an amount effective to modulate expression of the TLR gene in at least one of the cell populations.

The immune system includes various populations of cells, each population naturally expressing the different TLR genes to varying degrees. The various populations of cells populate different areas of the body including, but not limited to, the blood, skin, bone marrow, thymus, lymphatic system, and interstitial areas. For example, monocytes natively express relatively large amounts of TLR2 and TLR4, and also show significant levels of, for example, TLR1 and TLR8 expression. B lymphocytes exhibit relatively high native expression of TLR6, TLR7 and TLR9, but also express, for example, TLR1 and TLR10 to a lesser degree. Plasmacytoid dendritic cells (pDCs) predominantly express TLR7 and TLR9, but also express some TLR1 and TLR6.

With the discovery that some compounds may modulate expression of a TLR gene in one cell population and modulate expression of the same TLR gene in a different manner (qualitatively or quantitatively) in another cell population, the present invention provides means by which one can modulate the expression of a particular TLR gene selectively between different populations of cells of the immune system. The selective modulation of TLR gene expression between cells in different cell population may take the form of modulating TLR gene expression in one population of immune cells while leaving the expression of the same TLR gene in another population of immune cells substantially unmodulated (i.e., qualitative or “on-off” modulation). Alternatively, the selective modulation of TLR gene expression between cells in different cell populations may involve modulating the TLR gene expression in two or more immune cell populations to varying degrees (i.e., quantitative modulation).

For example, FIG. 5 in combination with FIG. 2 shows that a single compound (e.g., IRM1) can modulate expression of the same TLR gene in a qualitatively different manner in different cell types. FIG. 2 shows that IRM1 upregulates gene expression from the TLR3 gene in PBMCs, but FIG. 5 shows that IRM1 downregulates gene expression from the TLR3 gene in macrophages. Also, IRM3 and IRM5 upregulate gene expression from the TLR3 gene in PBMCs (FIG. 2), but do not significantly modulate gene expression from the TLR3 gene in macrophages (FIG. 5).

In certain embodiments, the methods of the present invention may include determining the TLR gene expression profile of the first cell population and the TLR gene expression profile of the second cell population. The TLR gene expression profile may be determined by any suitable method including, but not limited to, detection of TLR gene expression such as by PCR analysis, microarray gene analysis, or Northern blot analysis.

The modulation of TLR gene expression in any particular population of immune cells may include significantly upregulating TLR gene expression in the cells or significantly downregulating TLR gene expression in the cells. In some embodiments, at least a two-fold modulation of TLR gene expression may be considered significant, while less than a two-fold modulation of TLR gene expression may be considered insignificant. In other embodiments, at least a three-fold modulation of TLR gene expression may be considered significant, while less than a three-fold modulation of TLR gene expression may be considered insignificant. In still other embodiments, at least a four-fold modulation of TLR gene expression may be considered significant, while less than a four-fold modulation of TLR gene expression may be considered insignificant. The precise level of TLR gene expression modulation required to be considered significant may depend, at least in part, on factors including, but not limited to, the intended use of the identified compound (prophylactic, therapeutic, diagnostic, etc.); the quality (e.g., accuracy and/or precision) of the assay used to determine TLR gene expression; the particular cell populations and the native levels of expression of relevant TLR genes in the cells of those populations; and the environment in which the compound is intended to modulate TLR gene expression (e.g., in vitro or in vivo).

TLR gene expression may be modulated in selected cells by contacting the cells of the immune system with the selected compound either in vitro or in vivo. Modulating TLR gene expression in selected cells in vitro may include collecting a sample of immune cells from a subject, culturing the collected immune cells in vitro, and adding the selected compound to the cell culture. In some cases, the sample of immune cells collected from the subject may be a homogeneous sample of cells, i.e., the sample may include cells of only one population of immune cells. In other cases, the sample of immune cells collected from the subject may be a heterogeneous sample of cells, i.e., the sample may include cells of more than one population of immune cells. After the cells have been contacted with the selected compound so that TLR gene expression is modulated in selected cells, the treated cells (whether their TLR gene expression has been selectively modulated by contact with the selected compound or not) may be reintroduced into the subject, thereby providing prophylactic or therapeutic treatment. Alternatively, cells having their TLR gene expression selectively modulated in vitro may have diagnostic utility.

In some embodiments, cells selectively modulated in vitro may be genetically modified rather than collected from a subject. Such cells may have utility as experimental tools, such as, for example, further elucidating TLR-mediated biological activity.

In vivo modulation of TLR gene expression in selected cells may include administering the selected compound to a subject. The selected compound may be administered in any suitable manner including but not limited to topical, injection (e.g., intravenous, subcutaneous, intraperitoneal, intradermal), inhalation, ingestion, transdermal, or transmucosal delivery.

The particular amount of the selected compound effective for modulating TLR gene expression in selected immune cells in a subject may depend, at least in part, on one or more factors. Such factors include but are not limited to the particular compound being administered, the state of the subject's immune system (e.g., suppressed, compromised, stimulated); the identity and location of the cells whose TLR gene expression is being modulated; the route of administering the compound; the TLR gene expression profile of the cells whose TLR gene expression is being modulated; and the desired result (e.g., prophylactic or therapeutic treatment). Accordingly it is not practical to set forth generally the amount that constitutes an effective amount of compound. Those of ordinary skill in the art, however, can readily determine the appropriate amount with due consideration of such factors.

An amount of the selected compound effective to modulate TLR gene expression of selected immune cells is an amount sufficient to cause the targeted cell population or populations (e.g., monocytes, macrophages, dendritic cells, B cells, T cells, etc.) to alter expression of at least one TLR gene. The precise amount of selected compound effective for modulating TLR gene expression of selected immune cells will vary according to factors known in the art but in certain embodiments the amount can be a dose of from about 100 ng/kg to about 50 mg/kg, for example, from about 10 μg/kg to about 5 mg/kg. In other embodiments, the amount may be an amount sufficient to provide from about 0.001% to about 50% of the selected compound, by weight, in a suitable solution, suspension, emulsion, mixture, or the like. The minimum amount of the selected compound may vary, dependent upon the factors described above, but may be, in certain embodiments, 0.01%, 0.05%, 0.1%, 0.5%, or 1.0%. Similarly, the maximum amount of the selected compound may vary, dependent upon the factors described above, but may be, in certain embodiments, 1.0%, 2.0%, 5.0%, or 10%.

In some embodiments, the selected compound can be a known IRM compound including the small organic IRM molecules described below, or the purine derivatives, small heterocyclic compounds, amide derivatives, and oligonucleotide sequences described above. Alternatively, the selected compound may be a compound capable of selectively modulating expression of at least one TLR gene, identified by any suitable method of identifying such compounds, including some of the methods according to the present invention.

As noted above, a compound that selectively modulates expression of a TLR gene may be incorporated into a pharmaceutical composition. Such compositions may be useful for treatment of conditions treatable by selectively modulating expression of one or more TLR genes. A compound of the invention can be administered as the single therapeutic agent in the treatment regimen. Alternatively, a compound of the invention may be administered in combination with another compound of the invention or with one or more active agents including additional IRM compounds, immunogens, adjuvants, antivirals, antibiotics, etc.

Accordingly, the present invention also provides methods of treating a condition treatable by selectively modulating expression of a plurality of TLR genes. Generally, the methods include identifying a target TLR gene expression profile effective for treatment of the condition; selecting a compound having a TLR gene expression profile that conforms to the target TLR gene expression profile; and administering to the subject an amount of the compound effective for treating the condition.

Treating a condition may involve either prophylactic or therapeutic treatment. As used herein, prophylactic treatment refers to treatment initiated before the onset of symptoms or signs of the condition. Thus, prophylactic treatments generally are designed to: (1) reduce the likelihood that the subject receiving the treatment will acquire the condition, (2) reduce the severity of the condition, once acquired, or (3) both. As used herein, therapeutic treatment refers to treatment initiated after the onset of symptoms or signs of a condition. Thus, therapeutic treatments are designed to limit or reduce progression of the condition. In some cases, therapeutic treatments can result in reversal of the condition, even to the point of complete resolution.

In the methods of the invention, identifying the target TLR gene expression profile may involve determining which immune system cell population or populations might be well-suited for providing prophylactic or therapeutic treatment of the condition, then determining which TLR genes of the identified cell populations might be modulated to provide the desired treatment.

The TLR gene expression profile of the compound may be determined by performing one or more assays designed to detect modulation of TLR gene expression. Alternatively, the TLR gene expression profile of the IRM compound may be obtained from, for example, one or more published or unpublished sources.

Selecting a compound having a TLR gene expression profile that conforms to the target TLR gene expression profile involves the same considerations described above relating to assays for identifying a target compound having a particular TLR gene expression profile.

Conditions that may be treated using methods of the present invention include, but are not limited to:
(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picomavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);
(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;
(c) other infectious diseases, such chlamydia, fungal diseases including but not limited to candidiasis, aspergillosis, histoplasmosis, cryptococcal meningitis, or parasitic diseases including but not limited to malaria, pneumocystis camii pneumonia, leishmaniasis, cryptosporidiosis, toxoplasmosis, and trypanosome infection; and
(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, renal cell carcinoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers such as, for example, breast cancer, lung cancer, prostate cancer, colon cancer, etc.;
(e) TH2-mediated, atopic diseases, such as atopic dermatitis or eczema, eosinophilia, asthma, allergy, allergic rhinitis, and Ommen's syndrome;
(f) certain autoimmune diseases such as systemic lupus erythematosus, essential thrombocythaemia, multiple sclerosis, discoid lupus, alopecia greata; and
(g) diseases associated with wound repair such as, for example, inhibition of keloid formation and other types of scarring (e.g., enhancing wound healing, including chronic wounds).

Additionally, practicing certain embodiments of the invention may include using an IRM compound as a vaccine adjuvant in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens, toxoids, toxins; self-antigens; polysaccharides; proteins; glycoproteins; peptides; cellular vaccines; DNA vaccines; autologous vaccines; recombinant proteins; glycoproteins; peptides; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

Certain embodiments may be particularly helpful for providing treatment to individuals having compromised immune function. For example, certain embodiments may be used for treating the opportunistic infections and tumors that can occur after suppression of cell mediated immunity in, for example, transplant patients, cancer patients and HIV patients. As noted above, in certain aspects, the present invention includes pharmaceutical compositions that include a compound that selectively modulates TLR gene expression. The pharmaceutical composition may be administered in any suitable manner through any suitable delivery route. The compositions may be delivered topically or systemically. Suitable compositions for topical delivery include but are not limited to ointments, gels, foams, creams, lotions, solutions, suspensions, emulsions, pastes, powders, soaps, surfactant-containing cleaning preparations, solid sticks (e.g., wax- or petroleum-based sticks), oils and sprays. Typical systemic delivery routes include but are not limited to injection (e.g., intravenous, subcutaneous, intraperitoneal, intradermal), inhalation, ingestion, transdermal, or transmucosal delivery.

The compound may be provided in any formulation suitable for administration to a subject. Suitable types of formulations are described, for example, in U.S. Pat. No. 5,238,944; U.S. Pat. No. 5,939,090; U.S. Pat. No. 6,245,776; European Patent No. EP 0 394 026; and U.S. Patent Publication No. 2003/0199538. The compound may be provided in any suitable form including but not limited to a solution, a suspension, an emulsion, or any form of mixture. The compound may be delivered in formulation with any pharmaceutically acceptable excipient, carrier, or vehicle. The formulation may be delivered in any conventional topical dosage form including but not limited to a cream, an ointment, an aerosol formulation, a non-aerosol spray, a gel, a lotion, and the like. The formulation may further include one or more additives including but not limited to adjuvants, skin penetration enhancers, colorants, fragrances, moisturizers, thickeners, and the like.

In some embodiments, the methods of the present invention include administering the compound to a subject in a formulation of, for example, from about 0.001% to about 10% (unless otherwise indicated, all percentages provided herein are weight/weight with respect to the total formulation) to the subject, although in some embodiments the compound may be administered using a formulation that provides compound in a concentration outside of this range. In certain embodiments, the method includes administering to a subject a formulation that includes from about 0.01% to about 5% compound, such as, for example, a formulation that includes from about 0.1% to about 5% compound. In one particular embodiment, the method includes administering to a subject a formulation that includes 5% IRM compound.

An amount of a compound effective to treat a condition can vary according to factors known in the art including but not limited to the physical and chemical nature of the compound, the nature of the carrier, the intended dosing regimen, the state of the subject's immune system (e.g., suppressed, compromised, stimulated), the method of administering the compound, and the species to which the formulation is being administered. Accordingly it is not practical to set forth generally the amount that constitutes an amount of the compound effective to treat a condition for all possible applications. Those of ordinary skill in the art, however, can readily determine the appropriate amount with due consideration of such factors.

In some embodiments, the methods of the present invention include administering a sufficient amount of the compound to provide a dose of, for example, from about 100 ng/kg to about 50 mg/kg to the subject, although in some embodiments the methods may be performed by administering the compound in concentrations outside this range. In some of these embodiments, the method includes administering sufficient IRM compound to provide a dose of from about 10 μg/kg to about 5 mg/kg to the subject, for example, a dose of from about 100 μg/kg to about 1 mg/kg.

In some embodiments, the compound can be a known IRM compound including the small organic IRM molecules described in detail below, or the purine derivatives, small heterocyclic compounds, amide derivatives, and oligonucleotide sequences described above. Alternatively, the compound may be a compound capable of selectively modulating the expression of at least one TLR gene, identified by any suitable method of identifying such compounds, including some of the methods according to the present invention.

In some embodiments, suitable compounds include but are not limited to the small molecule IRM compounds described above. Suitable small molecule IRM compounds, having a 2-aminopyridine fused to a five membered nitrogen-containing heterocyclic ring, include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, a 6-, 7-, 8-, or 9-aryl, heteroaryl, aryloxy or arylalkyleneoxy substituted imidazoquinoline amine, or an imidazoquinoline diamine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

In some embodiments, the compound may be a tetrahydroimidazoquinoline amine such as, for example, 4-amino-2-(ethoxymethyl)-α,α-dimethyl-6,7,8,9-tetrahydro-1H-imidazo[4,5-c]quinoline-1-ethanol or a urea substituted tetrahydroimidazoquinoline amine such as, for example, N-[4-(4-amino-2-methyl-6,7,8,9,-tetrahydro-1H-imidazo[4,5-c]quinolin-1-yl)butyl]morpholine-4-carboxamide.

In other embodiments, the compound may be a thiazoloquinoline amine such as, for example, 2-propylthiazolo[4,5-c]quinolin-4-amine.

In other embodiments, the compound may be a sulfonamide substituted imidazoquinoline amine such as, for example, N-[4-(4-amino-2-butyl-1H-imidazo[4,5-c]quinolin-1-yl)butyl]methanesulfonamide, N-{4-[4-amino-2-(2-methoxyethyl)-1H-imidazo[4,5-c]quinolin-1-yl]butyl} methanesulfonamide, or N-[4-(4-amino-2-ethyl-1H-imidazo[4,5-c]quinolin-1-yl)butyl]methane sulfonamide.

In other embodiments, the compound may be an imidazoquinoline amine such as, for example, 1-(2-methylpropyl)-1H-imidazo[4,5-c]quinolin-4-amine or 4-amino-α,α,2-trimethyl-1H-imidazo[4,5-c]quinoline-1-ethanol.

In other embodiments, the compound may be an imidazonaphthyridine amine such as, for example, 1-(2-methylpropyl)-1H-imidazo[4,5-c][1,5]naphthyridin-4-amine or 2-methyl-1-(2-methylpropyl)-1H-imidazo[4,5-c][1,5]naphthyridin-4-amine.

In still other embodiments, the compound may be a sulfonamide substituted imidazopyridine amine such as, for example, N-[4-(4-amino-2-butyl-6,7-dimethyl-1H-imidazo[4,5-c]pyridin-1-yl)butyl]methanesulfonamide.

The methods of the present invention may be performed on any suitable subject. Suitable subjects include but are not limited to animals such as but not limited to humans, non-human primates, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

IRM Compounds

The IRM compounds used in the examples are shown in Table 1.

TABLE 1

| Compound | Chemical Name | Reference |
|---|---|---|
| IRM1 | 4-amino-2-(ethoxymethyl)-α,α-dimethyl-6,7,8,9- | U.S. Pat. No. 5,352,784 |
| | tetrahydro-1H-imidazo[4,5-c]quinoline-1-ethanol | Example 91 |
| IRM2 | 2-propylthiazolo[4,5-c]quinolin-4-amine | U.S. Pat. No. 6,110,929 |
| | | Example 12 |
| IRM3 | N-[4-(4-amino-2-butyl-1H-imidazo[4,5-c]quinolin-1- | U.S. Pat. No. 6,331,539 |
| | yl)butyl]methanesulfonamide | Example 6 |
| IRM4 | N-[4-(4-amino-2-methyl-6,7,8,9,-tetrahydro-1H- | U.S. Pat. No. 6,573,273 |
| | imidazo[4,5-c]quinolin-1-yl)butyl]morpholine-4- | Example 170 |
| | carboxamide | |
| IRM5 | 1-(2-methylpropyl)-1H-imidazo[4,5-c]quinolin-4-amine | U.S. Pat. No. 4,689,338 |
| | | Example 99 |
| IRM6 | 4-amino-α,α,2-trimethyl-1H-imidazo[4,5-c]quinoline-1- | U.S. Pat. No. 5,266,575 |
| | ethanol | Example C1 |
| IRM7 | 1-(2-methylpropyl)-1H-imidazo[4,5-c] | U.S. Pat. No. 6,194,425 |
| | [1,5]naphthyridin-4-amine | Example 32 |
| IRM8 | 2-methyl-1-(2-methylpropyl)-1H-imidazo[4,5-c] | U.S. Pat. No. 6,194,425 |
| | [1,5]naphthyridin-4-amine | Example 36 |
| IRM9 | N-{4-[4-amino-2-(2-methoxyethyl)-1H-imidazo[4,5- | U.S. Pat. No. 6,331,539 |
| | c]quinolin-1-yl]butyl}methanesulfonamide | Example 111 |
| IRM10 | N-[4-(4-amino-2-butyl-6,7-dimethyl-1H-imidazo[4,5- | U.S. Pat. No. 6,525,064 |
| | c]pyridin-1-yl)butyl]methanesulfonamide | Example 2 |
| IRM11 | N-[4-(4-amino-2-ethyl-1H-imidazo[4,5-c]quinolin-1- | U.S. Pat. No. 6,677,349 |
| | yl)butyl]methanesulfonamide | Example 236 |

### Example 1

For some experiments, Peripheral Blood Mononuclear Cells (PBMCs) were isolated from donors by Ficol gradient centrifugation. For other experiments, leukophoresed mononuclear cells were obtained from AllCells, LLC (Berkeley, Calif.). Monocyte derived macrophages and dendritic cells were prepared from PBMCs using positive selection of CD14+ cells with Miltenyi micro beads (Miltenyi Biotec Inc., Auburn, Calif.). Dendritic cells were differentiated in RPMI 1640 with 10% fetal bovine serum, using 33 ng/mL IL4 and 66 ng/mL GM-CSF, and macrophages were differentiated using 25 ng/mL M-CSF, for seven days at 37° C.

IRM compounds were prepared as 1000× stocks in DMSO. PBMCs were diluted to approximately 3.0×10⁶ cells per mL in Ex-Vivo 20 media, and 1.0 mL was distributed per well in a 96 deep well plate. Cells were allowed to equilibrate at 37° C. for 1 hour, then IRM compound was added to the culture.

At each indicated time point, cells were harvested as follows. PBMCs and dendritic cells were harvested by centrifuging the plate at 1500 RPM, 360 RCF, at 4° C. in a Qiagen SIGMA centrifuge (Qiagen Inc., Valencia, Calif.). The media was then vacuum aspirated and 400 μL of RLT buffer with 1.0% 2-mercaptoethanol was added to each well. Macrophage cultures were harvested by aspiration of the media and direct addition of the RLT buffer to the well. The total RNA was purified using a semi-automated procedure on the Qiagen BioRobot 8000 (Qiagen Inc., Valencia, Calif.), with an incorporated DNase digestion step.

Purified total RNA was transferred to Costar 3565 plate and the optical density at 260 nm and 280 nm was read using a Molecular Devices SpectroMax 384 Plus (Molecular Devices Corp., Sunnyvale, Calif.). The RNA was then ethanol precipitated, washed, and the pellet re-suspended in 10 μL of water. cDNA was made with the Invitrogen Superscript II Kit (Invitrogen Corp., Carlsbad, Calif.) using random priming and 2-3 μg of total RNA. PCR was performed using an ABI 7900 (Applied Biosystems Corp., Foster City, Calif.), and the 384 well Microfluidic Card with Taqman™ chemistry, and the reactions were standardized using 2 ng/μL cDNA in the master mix. Cycling conditions were 50° C. for 10 minutes, 95° C. for 2 minutes, then 35 cycles of 95° C. for 30 seconds, and 60° C. for 1 minute. Data was analyzed using SDS 2.0 software (Applied Biosystems, Inc.) using a threshold value of 0.1. The results were imported into Excel and the expression fold changes were calculated using the ΔΔCt method, (User Bulletin #2, PE Applied BioSystems, Inc.). Normalization of signals was performed using the housekeeping gene, GAPDH.

### Example 2

A phase II, double-blind, vehicle-controlled, randomized, parallel group study included 17 male subjects with histologically confirmed actinic keratosis (AK). Eligible subjects were randomized to receive either IRM5 formulated as a 5% cream (ALDARA, 3M Pharmaceuticals, St. Paul, Minn.) or vehicle cream in a 3:1 ratio. Study subjects had at least five clinically typical, discrete, visible AK lesions within a 25 cm² area on the balding scalp that were suitable for shave biopsies.

Subjects applied 1 sachet (250 mg) of study cream to the treatment area 3 times per week for 4 weeks. Each dose of study cream was applied prior to normal sleeping hours at approximately the same time on each dosing day and was to remain on the skin for approximately 8 hours.

Each subject could have up to 8 shave biopsies performed. Biopsy samples obtained at the treatment initiation visit and all subsequent visits were designated for gene expression analysis. At the treatment initiation visit (T=0), an intact lesion in the treatment area was biopsied (sample series A) to establish a baseline for AK gene expression. Also at this visit, a sun-exposed area of the scalp located outside the treatment area that did not contain lesions was biopsied (sample series B), as was a sun-unexposed area of the body that did not contain lesions (sample series C). The sun-unexposed area biopsy was used to establish baseline control for gene expression.

A biopsy of one of the remaining lesions in the treatment area was taken after 1 week of treatment (sample series D), after 2 weeks of treatment (sample series E), after four weeks of treatment (sample series F), and four weeks after completion of four weeks of treatment (i.e., T=8, (sample series G)).

TABLE 2

| | Time | | |
|---|---|---|---|
| Series | (weeks) | Biopsy Target | Location |
| A | 0 | Untreated AK lesion | Treatment area |
| B | 0 | Non-AK (sun-exposed) | Outside of treatment area |
| C | 0 | Non-AK (unexposed) | Outside of treatment area |
| D | 1 | IRM-treated AK Lesion | Treatment area |
| E | 2 | IRM-treated AK Lesion | Treatment area |
| F | 4 | IRM-treated AK Lesion | Treatment area |
| G | 8 | IRM-treated AK Lesion | Treatment area |

RNA from each sample was extracted and expression of TLR1, TLR3, TLR6, TLR7, TLR8, TLR9, and TLR10 was analyzed as described in Example 1. The highest response time point (i.e., highest response among Series D, E, F and G) was used to compute the change in expression of the indicated TLRs with respect to Series C (non-AK skin from sun-unexposed area).

ANOVA (ANalysis Of VAriance) was performed on the data to determine differences in fold change between untreated AK lesions and IRM5-treated AK lesions. P-Value ≦0.05 signify statistically significant differences in TLR expression between untreated AK biopsy samples and IRM-treated AK biopsy samples. Results are shown in Table 3.

TABLE 3

| | Median Fold | Median Fold Change - | |
|---|---|---|---|
| | Change AK | IRM5-treated | P- |
| TLR | (Series A/Series C) | (IRM-treated/Series C) | Value |
| 1 | 1.1 | 2.1 | 0.010 |
| 3 | −2.1 | 1.5 | 0.047 |
| 6 | −1.1 | 1.9 | 0.002 |
| 7 | −1.0 | 4.9 | 0.037 |
| 8 | −1.8 | 2.4 | 0.005 |
| 9 | 2.9 | 15.2 | 0.006 |
| 10 | 1.3 | 3.6 | 0.041 |

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A method of identifying a compound that selectively modulates expression of at least one TLR gene, the method comprising:
(1) providing an assay to detect expression of each of a plurality of TLR genes;
(2) performing each assay using a test compound; and
(3) identifying the test compound as a compound that selectively modulates expression of at least one TLR gene if the test compound modulates expression of a first TLR gene to a different extent than it modulates expression of at least one second TLR gene.

2. The method of claim 1 wherein the compound induces expression of a first TLR gene and does not induce expression of at least one second TLR gene.

3. A compound identified according to the method of claim 1, and salts thereof.

4. A pharmaceutical composition comprising a compound identified according to the method of claim 1, a pharmaceutically acceptable salt thereof, a derivative thereof, or a pro-drug thereof.

5. A method of identifying a target compound having a target TLR gene expression profile, the method comprising:
(1) selecting a target TLR gene expression profile;
(2) determining the TLR gene expression profile of a test compound; and
(3) identifying the test compound as a target compound if the TLR gene expression profile of the test compound includes the target TLR gene expression profile.

6. The method of claim 5 wherein the target TLR gene expression profile includes one or more TLR genes that are not detectably induced by a target compound.

7. The method of claim 5 wherein determining the TLR gene expression profile of a test compound comprises performing at least one assay for detecting expression of a TLR gene.

8. A compound identified as a target compound according to the method of claim 5 or a pharmaceutically acceptable form thereof.

9. A pharmaceutical composition comprising a target compound identified according to the method of claim 5, a pharmaceutically acceptable form thereof, a derivative thereof, or a pro-drug thereof.

10. A method of modulating expression of a TLR gene in a selected population of cells of the immune system, the method comprising:
(1) identifying a first immune system cell population and a second immune system cell population;
(2) selecting a compound that modulates expression of a TLR gene of the first cell population to a different extent than it modulates expression of the same TLR gene in the second cell population; and
(3) contacting cells of the immune system with the selected compound in an amount effective to modulate expression of at least one TLR gene in at least one of the cell populations.

11. The method of claim 10 wherein at least one cell population is contacted with the selected compound in vitro.

12. The method of claim 10 wherein at least one cell population is contacted with the selected compound in vivo.

13. A method of treating a condition treatable by selectively modulating expression of at least one of a plurality of TLR genes in a subject, the method comprising:
(1) identifying a target TLR expression profile effective for treatment of the condition;
(2) selecting a compound having a TLR expression profile that conforms to the target profile; and
(3) administering to the subject an amount of the compound effective for treating the condition.

14. The method of claim 13 wherein the condition is an infectious disease or a neoplastic condition.

15. The method of claim 14 wherein the infectious disease is a viral disease, a fungal disease, a parasitic disease, a bacterial disease, or a prion-mediated disease.

16. The method of claim 14 wherein the neoplastic condition is an intraepithelial neoplasm, a pre-cancerous neoplasm, or a cancer.
